(19) United States
(12) Patent Application Publication
(10) Pub. No.: US 2015/0182125 A1
(43) Pub. Date: Jul. 2, 2015

(54) PHOTOACOUSTIC APPARATUS, SIGNAL PROCESSING METHOD, AND PROGRAM

(71) Applicant: CANON KABUSHIKI KAISHA, Tokyo (JP)

(72) Inventor: Kazuhiko Fukutani, Kyoto-shi (JP)

(21) Appl. No.: 14/570,745

(22) Filed: Dec. 15, 2014

(30) Foreign Application Priority Data
Dec. 26, 2013 (JP) 2013-269691

(51) Int. Cl. A61B 5/00; G01N 29/24; G01N 29/44

(52) U.S. Cl. CPC A61B 5/0095; G01N 29/4463; G01N 29/2418; A61B 5/742; G01N 2291/011; G01N 2291/023

(57) ABSTRACT

An apparatus includes a receiving unit configured to receive a photoacoustic wave that is generated when an object is illuminated with light and to output a time-series received signal, and a processing unit configured to acquire object information by performing a reconstruction process on the time-series received signal using a specific speed of sound, wherein in the reconstruction process, the processing unit converts a reception time of the time-series received signal to a reception time normalized with respect to the specific speed of sound.

### BACKGROUND OF THE INVENTION

1. Field of the Invention

The present invention relates to a photoacoustic apparatus that acquires object information using a received signal of a photoacoustic wave generated when an object is illuminated with light.

2. Description of the Related Art

Intensive research is being made on an optical imaging apparatus for use in a medical field. In the optical imaging apparatus, a living body is illuminated with light emitted from a light source, such as a laser, and information obtained based on the incident light is acquired in the form of an image. Photoacoustic imaging (PAI) is known as one of such optical imaging techniques. In the photoacoustic imaging, when a living body is illuminated with light generated by a light source, energy of the light is absorbed by a body tissue when the light propagates or is scattered in the living body, and correspondingly an acoustic wave (typically, an ultrasonic wave) is generated by the body tissue. More specifically, when there is a difference in optical energy absorption rate between a part to be inspected such as a tumor and other tissues, absorption of the illuminating optical energy by the part to be inspected may cause the part to be inspected to instantaneously expand, which may generate an elastic wave. The elastic wave is received by an acoustic wave receiving unit (also referred to as a probe or a transducer). By performing an analysis process on this received signal, it is possible to obtain an image corresponding to an initial pressure distribution or an absorbed optical energy density distribution (the product of an absorption coefficient distribution and a light fluence distribution) (“Photoacoustic imaging in biomedicine”, M. Xu, L. V. Wang, REVIEW OF SCIENTIFIC INSTRUMENT, 77, 041101, 2006). By employing various wavelengths for the light used in acquiring the image information, it is possible to perform a quantitative measurement of a specific substance in the object, for example, a density of hemoglobin contained in blood, oxygen saturation of blood, and the like. In recent years, using the above-described photoacoustic imaging, a preclinical study on imaging a blood vessel of a small animal has been intensively performed. Furthermore, a clinical study for applying the photoacoustic imaging to diagnosis of breast cancer, prostate cancer, carotid plaque, and the like has also been intensively performed.

In the photoacoustic imaging, a diagnostic image is formed based on object information by performing a reconstruction process on a received signal taking into account a speed of sound in a propagation path of an acoustic wave.

### SUMMARY OF THE INVENTION

According to an aspect of the disclosure, an apparatus includes a receiving unit configured to receive a photoacoustic wave that is generated when an object is illuminated with light and to output a time-series received signal, and a processing unit configured to acquire object information by performing a reconstruction process on the time-series received signal using a specific speed of sound, wherein in the reconstruction process, the processing unit converts a reception time of the time-series received signal to a reception time normalized with respect to the specific speed of sound.

Further features of the present invention will become apparent from the following description of exemplary embodiments with reference to the attached drawings.

### BRIEF DESCRIPTION OF THE DRAWINGS

FIG. 1 is a schematic diagram illustrating an example of a photoacoustic apparatus according to an embodiment.

FIG. 2 is a schematic diagram illustrating connections of elements of a photoacoustic apparatus according to an embodiment.

FIG. 3A is a diagram illustrating an example of a time-series received signal obtained by a photoacoustic apparatus according to an embodiment.

FIG. 3B is a schematic diagram illustrating a relative positional relationship between an object and an acoustic wave receiving unit of a photoacoustic apparatus according to an embodiment.

FIG. 4 is a flow chart illustrating an example of an operation of a photoacoustic apparatus according to an embodiment.

FIG. 5A is a diagram illustrating an example of an image obtained by a photoacoustic apparatus according to a first embodiment.

FIG. 5B is a diagram illustrating an image obtained in a comparable example.

FIG. 6 is a schematic diagram illustrating an example of a photoacoustic apparatus according to a second embodiment.

### DESCRIPTION OF THE EMBODIMENTS

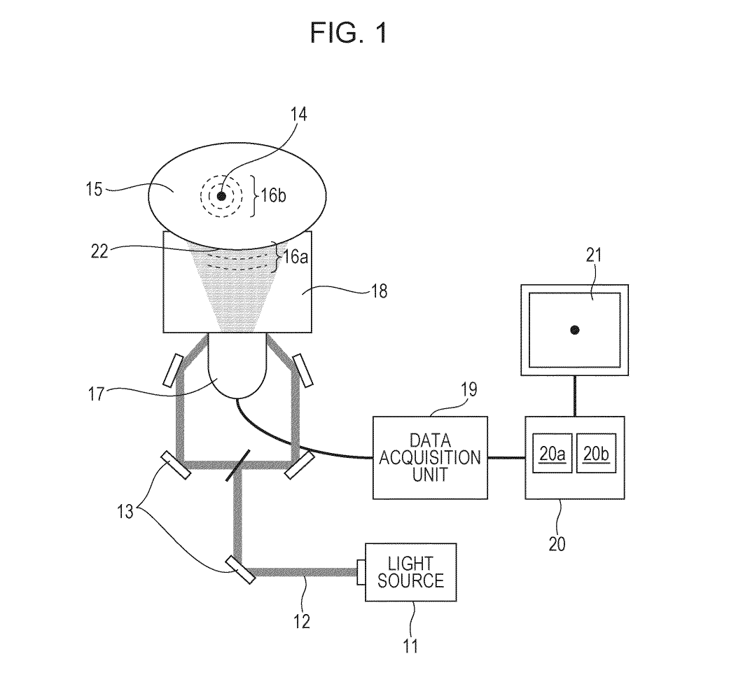

Referring to FIG. 1, a basic configuration of a photoacoustic apparatus according to an embodiment is described below. The photoacoustic apparatus according to the present embodiment is an apparatus configured to acquire object information associated with an inside of an object. In the present embodiment, the object information refers to optical characteristic value such as an initial sound pressure distribution, an absorbed optical energy density distribution, or an absorption coefficient distribution obtained therefrom. Note that in the present embodiment, the object information includes a distribution of a substance included in an object obtained from a plurality of absorption coefficient distributions for a plurality of wavelengths.

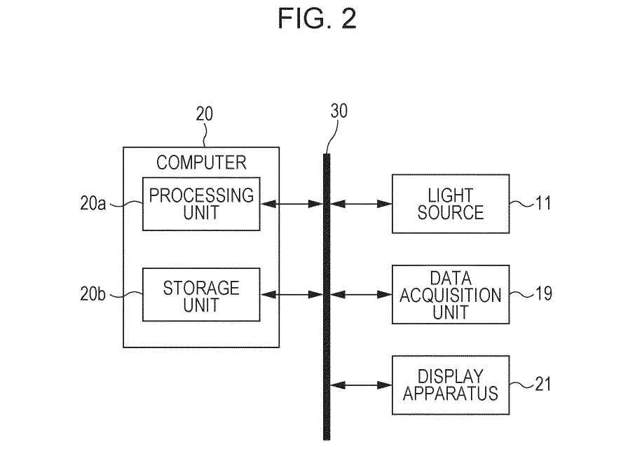

A basic hardware configuration of the photoacoustic apparatus according to the present embodiment includes a light source 11, an optical system 13, an acoustic wave receiving unit 17, an acoustic matching material 18, a data acquisition unit 19, a computer 20 functioning as a processing unit, and a display apparatus 21. The computer 20 includes a processing unit 20a and a storage unit 20b. As illustrated in FIG. 2, the processing unit 20a controls, via a bus 30, operations of elements of the photoacoustic apparatus.

Light 12 emitted from the light source 11 is transmitted while being formed into a particular shape via the optical system 13 which may include, for example, a lens, a mirror, an optical fiber, a diffusing plate and the like such that an object 15 such as a living body is illuminated with the light 12. When part of energy of the light propagating in the object 15 is absorbed by a light absorbent 14 (functioning as a sound source when absorbing light) such as a blood vessel, the light absorbent 14 is thermally expanded, which causes photoacoustic waves (typically ultrasonic waves) 16a and 16b to be generated. The acoustic wave receiving unit 17 receives the photoacoustic waves 16a and 16b and outputs a time-series received signal. The data acquisition unit 19 performs processing such as amplification, analog-to-digital conversion, and the like on the output time-series received signal and stores the resultant time-series received signal in the form of a digital signal in the storage unit 20b. The processing unit 20a generates object information by performing a signal processing on the time-series received signal stored in the storage unit 20b. The generated object information is displayed in the form of an image or numerical data on the display apparatus 21.

The signal processing performed by the photoacoustic apparatus according to the present embodiment is described below.

### <Signal Processing Method>

In the present embodiment, when the photoacoustic apparatus acquires object information by performing a reconstruction process on the time-series received signal, the photoacoustic apparatus converts a reception time of the time-series received signal to a reception time normalized with respect to a specific speed of sound. That is, time-sampled data corresponding to each reception time is treated as time-sampled data corresponding to a received signal normalized with respect to the specific speed of sound. In a case where the time-series received signal is treated in the above-described manner, a value obtained by multiplying the reception time by the specific speed of sound represents a distance between the acoustic wave receiving unit 17 and a position where a photoacoustic wave corresponding to time-sampled data is generated at that reception time.

Therefore, in the photoacoustic apparatus according to the present embodiment, when the reconstruction process is performed assuming that the speed of sound of the photoacoustic wave is equal to the specific speed of sound for any propagation path, it is possible to suppress an influence of a difference between the speed of sound in the object 15 and the speed of sound in the acoustic matching material 18. That is, in the photoacoustic apparatus according to the present embodiment, even in a case where the reconstruction process is performed on the time-series received signal using only the specific speed of sound, it is possible to acquire object information while suppressing the influence of the difference between the speed of sound in the object 15 and the speed of sound in the acoustic matching material 18.

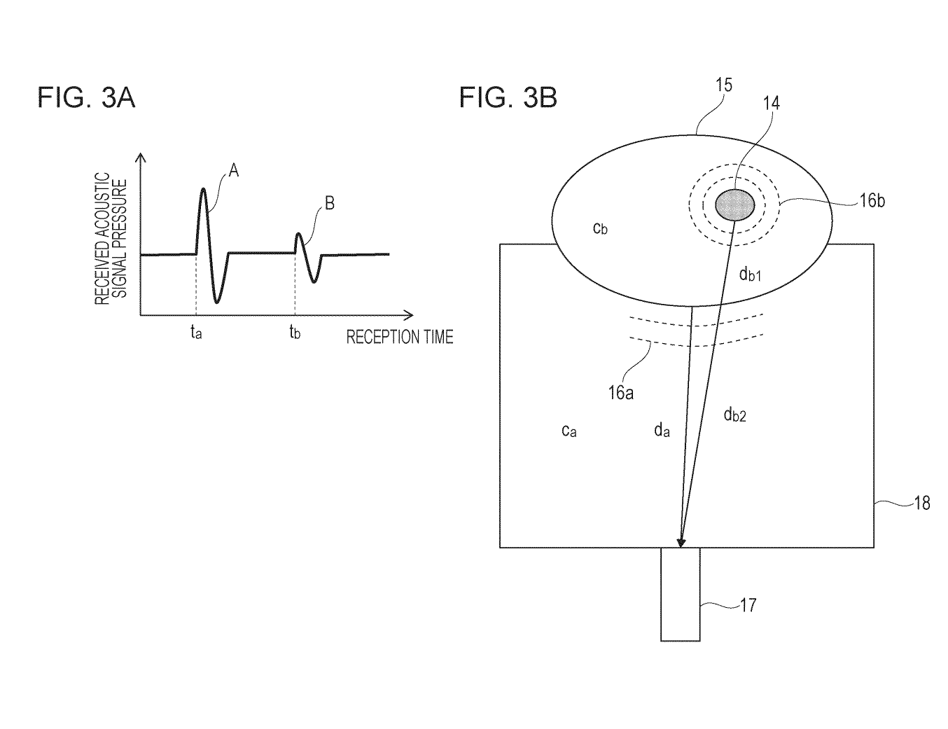

The signal processing method according to the present embodiment is described in further detail below with reference to FIGS. 3A and 3B.

FIG. 3A is a diagram illustrating typical received signal data measured at various times (time-series received signal) received by the acoustic wave receiving unit 17. In FIG. 3A, a horizontal axis represent the reception time. A zero point on the horizontal axis indicates a time at which light hits the object. A vertical axis represents a value proportional to a sound pressure received by the acoustic wave receiving unit 17.

Typically, the acoustic wave receiving unit 17 receives photoacoustic waves generated at different positions in the object 15. For example, a signal A and a signal B in FIG. 3A are received signals of photoacoustic waves generated at different positions. In a case where the object 15 is a living body, the living body generally includes much melanin in a region close to an epidermis. The melanin has a high light absorption rate, and thus a photoacoustic wave with a large amplitude is generated in the region close to the epidermis of the living body. Therefore, in the system configuration illustrated in FIG. 1, a photoacoustic wave 16a generated on a surface 22 of the object 15 as illustrated in FIG. 1 is first received by the acoustic wave receiving unit 17 and observed as the signal A as illustrated in FIG. 3A. Subsequently, a photoacoustic wave 16b generated by a light absorbent 14 (which will be reconstructed later) inside the object 15 is received and observed as the signal B as illustrated in FIG. 3A.

FIG. 3B illustrates a positional relationship between the acoustic wave receiving unit 17 and the object 15. As may be seen from FIG. 3B, the signal A in FIG. 3A originates from the photoacoustic wave 16a that does not propagate through the object 15 but propagates through only the acoustic matching material 18. The signal A in FIG. 3A is received at a reception time tₐ given by a value obtained dividing a shortest distance dₐ between the acoustic wave receiving unit 17 and the surface 22 of the object 15 by the speed of sound cₐ of the acoustic matching material 18. That is, the reception time tₐ is represented by equation (1) described below.

tₐ=dₐ/cₐ (1)

When the distance between the surface 22 of the object 15 and the acoustic wave receiving unit 17 is denoted by d_{b2}, and the distance between the surface 22 of the object 15 and the light absorbent 14 in the object 15 is denoted by d_{b1}, a reception time t_{b} of the signal B in FIG. 3A is given by the sum of a value obtained by diving d_{b2} by cₐ and a value obtained by dividing d_{b1} by c_{b}. That is, the reception time t_{b} is represented by equation (2) described below.

t_{b}=d_{b2}/cₐ+d_{b1}/c_{b} (2)

By approximating d_{b2} as d_{b2}=dₐ, the reception time t_{b} is represented by equation (3) as follows.

t_{b}=dₐ/cₐ+d_{b1}/cb=tₐ+d_{b1}/c_{b} (3)

That is, equation (3) provides an approximate value of t_{b} assuming that the distance between surface 22 of the object 15 and the acoustic wave receiving unit 17 is constant and given by dₐ.

The approximation by d_{b2}=dₐ becomes better when the acoustic wave receiving unit 17 and the object 15 are relatively positioned such that the condition d_{b1}<d_{b2} is satisfied. As the approximation by d_{b2}=dₐ becomes better in the positional relationship between the object 15 and the acoustic wave receiving unit 17, equation (3) provides higher approximation accuracy, and object information (described later) obtained using t_{b} represented by equation (3) provides higher accuracy. Therefore, a good result is obtained by disposing the acoustic wave receiving unit 17 such that d_{b1}<d_{b2}. Note that d_{b1} denotes the distance between the surface 22 of the object 15 and a smallest constituent unit (a pixel or a voxel) to be reconstructed, that is, a virtual sound source. To achieve the condition d_{b1}<d_{b2}, it may be desirable to select the acoustic matching material 18 having a thickness greater than the distance between the surface 22 of the object 15 and a smallest constituent unit located farthest from the surface 22 of the object 15. In a specific example in which the object 15 is a breast, the condition d_{b1}<d_{b2} is easily achieved by placing the acoustic wave receiving unit 17 and the object 15 such that the distance between the acoustic wave receiving unit 17 and the surface of the object 15 is equal to or greater than 50 mm for a typical size of the breast.

From equation (1), the distance dₐ between the acoustic wave receiving unit 17 and the surface 22 of the object 15 is expressed by equation (4) as described below.

dₐ=cₐ×tₐ (4)

On the other hand, the distance d_{b} between the acoustic wave receiving unit 17 and the light absorbent 14 in the object 15 is expressed by equation (5) described below.

d_{b}=(cₐ×tₐ)+(t_{b}−tₐ)×c_{b} (5)

Here if tₐ′ is introduced such that tₐ′=tₐ×cₐ/c_{b}, then the distance db is expressed by equation (6) described below.

d_{b}=(ta′+t_{b}−tₐ)×c_{b} (6)

According to equation (6), the distance d_{b} is described only by multiplying time t_{b}′=(ta′+t_{b}−tₐ) expressed using tₐ′=tₐ×cₐ/c_{b} by the speed of sound c_{b}.

In the present embodiment, in view of the above, each reception time t1 of time-sampled data of the time-series received signal in a period until a reception time tₐ is converted to t2=t1×ca/cb. Furthermore, each reception time t1 of time-sampled data of the time-series received signal in a period after the reception time tₐ is converted to t2=tₐ×cₐ/c_{b}+(t1−tₐ). Using the time-series received signal and the converted data of the reception time, the reconstruction process is performed using only the speed of sound c_{b} in the object 15 thereby acquiring object information. That is, the reconstruction process is performed assuming that the speed of sound in any propagation path of the photoacoustic wave is equal to the speed of sound c_{b} in the object 15. By performing reconstruction process in the above-described manner, it is possible to obtain subject information while suppressing the influence of the difference between the speed of sound in the subject 15 and the speed of sound in the acoustic matching material 18. Furthermore, because the reconstruction process is performed assuming that the speed of sound in any propagation path of the photoacoustic wave is equal to the speed of sound in the subject 15, it is possible to performed the reconstruction process for a shorter time than in the case where the reconstruction process is performed taking account the sound speed distribution.

The reconstruction process may be alternatively performed such that each reception time t1 of the time-sampled data of the time-series received signal in a period after the reception time tₐ is converted to t2=tₐ+(t1−tₐ)×c_{b}/cₐ, and the speed of sound cₐ in the acoustic matching material 18 is used in the reconstruction process. Also in this case, it is possible to obtain subject information while suppressing the influence of the difference between the speed of sound in the subject 15 and the speed of sound in the acoustic matching material 18.

In the present specification, the reception time t1 of the time-series received signal acquired by the acoustic wave receiving unit 17 is referred to as a first reception time, and the reception time t2 obtained as a result of the conversion with respect to the specific speed of sound is referred to as a second reception time.

As for the speed of sound in the object 15 and the speed of sound in the acoustic matching material 18, empirical values, values described in literature, or measured values or the like may be used. As for the speed of sound, typically, an average speed of sound in the material may be used.

### <Detailed Description of Elements of Photoacoustic Apparatus>

Elements of the photoacoustic apparatus according to the present embodiment are described below.

### (Light Source 11)

In a case where the object 15 is a living body, it may be desirable that the light emitted from the light source 11 to illuminate the living body has a wavelength selectively absorbed by a particular component of the living body. The light source 11 may be provided in an integral form with the photoacoustic apparatus according to the present embodiment or may be provided separately from the photoacoustic apparatus according to the present embodiment. As for the light source 11, it may be desirable to use a laser capable of providing large optical output power. Instead of the laser, a light emitting diode or the like may be used. Examples of lasers usable as the light source 11 include a solid-state laser, a gas laser, a dye laser, a semiconductor laser, and the like. More specifically, for example, an OPO laser, a dye laser, or a Ti:Sa laser, which may be pumped by a YAG laser, may be employed. As for the wavelength of light used, it may be desirable to employ a wavelength that allows the light to propagate into the object 15. More specifically, in the case where the object 15 is a living body, it may be desirable to employ a wavelength in a range larger than or equal to 500 nm and smaller than or equal to 1200 nm. As for the light source 11, it may be desirable to employ a pulsed light source capable of generating pulsed light with a pulse width in a range from several nanoseconds to several hundred nanoseconds. The light source 11 may include a plurality of light sources.

### (Optical System 13)

The optical system 13 has a function of transmitting and shaping the light 12 emitted from the light source 11 such that the light 12 has a particular shape and the surface of the object is illuminated with the light 12. To make it possible to transmit the light 12 into a specified particular direction, the optical system 13 may include a mirror, an optical fiber, and/or the like. To make it possible to obtain a specified particular illumination pattern, the optical system 13 may include, for example, a diffusing plate, a lens, and/or the like.

The optical system 13 may be configured such that the light 12 from the light source 11 is directed from the side of the acoustic wave receiving unit toward the object 15. By performing the illumination in this manner, the received signal corresponding to the photoacoustic wave 16a generated at the surface 22 of the object 15 is first observed, which makes it easy to detect the received signal corresponding to the photoacoustic wave 16a. Note that in a case where it is possible to obtain a specified particular light illumination pattern at a specified particular location on the object 15 by illuminating the object 15 directly with the light 12 emitted from the light source 11, the optical system 13 may be unnecessary.

### (Light Absorbent 14 and Object 15)

Next, the light absorbent 14 and the object 15 are described below although they are not elements of the photoacoustic apparatus. The photoacoustic apparatus according to the present embodiment is supposed to be used, for example, for angiography, diagnosis of a malignant tumor or a blood vessel disease of a human or an animal, monitoring of an effect of a chemical treatment, and the like. The object 15 may be a living body, and more specific examples of objects to be subjected to diagnosis include a breast, a finger, a limb, or the like, of a human or an animal. In a case where the object 15 is a small animal such as a mouse, not only a particular part thereof but a whole small animal may be an object to be observed.

It may be desirable that the light absorbent 14 located inside the object 15 has a high relative absorption coefficient in the object 15. In a case where the object 15 to be observed is a human body, examples of light absorbents 14 having high absorption coefficients are oxyhemoglobin, deoxyhemoglobin, and the like, although depending on the wavelength of the light 12 used. A blood vessel containing oxyhemoglobin and deoxyhemoglobin also functions as a light absorbent 14. A malignant tumor having a new blood vessel also functions as a light absorbent 14. Melanin existing close to the surface of a skin functions as a light absorbent 14 on the surface 22 of the object 15. The light absorber 14 may be a substance introduced from the outside, for example, a pigment such as methylene blue (MB), indocyanine green (ICG), or the like, or a gold fine particle, or an integrated or chemically-modified substance thereof.

### (Acoustic Wave Receiving Unit 17)

The acoustic wave receiving unit 17 functioning as a receiver that receives a photoacoustic wave generated on the surface 22 of or inside the object 15 in response to excitement by the light 12 is a transducer configured to receive the photoacoustic wave and convert the received photoacoustic wave into an analog electric signal. Hereinafter, the acoustic wave receiving unit will also be referred to as a probe or a transducer. The acoustic wave receiving unit 17 may be a transducer using a piezoelectric phenomenon, a transducer using optical resonance, a transducer using a capacitance change, or any other types of transducers as long as they are capable of receiving acoustic waves.

In the present embodiment, the acoustic wave receiving unit 17 typically includes a plurality of receiving elements disposed in a one-dimensional, two-dimensional, or three-dimensional manner. From the point of view of the principle of the reconstruction, it may be desirable to dispose the plurality of receiving elements in a flat plane, on a circular cylinder surface, on a spherical surface, or the like or on a part thereof. Use of the receiving elements arranged in such a multidimensional array makes it possible to receive acoustic waves simultaneously at a plurality of positions, which allows it to reduce the measurement time. The reduction in the measurement time makes it possible to reduce effects of vibration of the object or the like. However, alternatively, the acoustic wave receiving unit 17 may include only one receiving element and the acoustic wave receiving unit 17 may be moved to receive acoustic waves at a plurality of locations without providing a plurality of receiving elements in the one-dimensional, two-dimensional, or three-dimensional manner on the acoustic wave receiving unit 17. Note that when the acoustic wave receiving unit 17 including receiving elements arranged in the multidimensional array is used, the acoustic wave receiving unit 17 may be moved to receive acoustic wave at further various positions thereby achieving further improved image quality.

### (Acoustic Matching Material 18)

The acoustic matching material 18 is a material disposed between the object 15 and the acoustic wave receiving unit 17 and is used to achieve acoustic matching between the acoustic wave receiving unit 17 and the object 15. In general, the acoustic matching material 18 is realized using a material having acoustic impedance between the acoustic impedance of the object 15 and the acoustic impedance of the acoustic wave receiving unit 17. More specifically, it may be desirable to employ a material having an acoustic impedance close to that of the object 15. As for the material of the acoustic matching material 18, it may also be desirable that the shape of the material is flexibly changed according to the shape of the object 15 such that an undesirable gap between the object 15 and the acoustic wave receiving unit 17 is minimized. More specifically, in the case where the object 15 is a living body, the acoustic matching material 18 may be water, ultrasonic gel, a gel-like material containing water or similar constituent, or the like. Note that the acoustic matching material 18 may be provided separately from the photoacoustic apparatus.

### (Data Acquisition Unit 19)

The data acquisition unit 19 amplifies the received signal output from the acoustic wave receiving unit 17 and converts the amplified received signal from an analog form into a digital signal. The data acquisition unit 19 may typically include an amplifier, an analog-to-digital converter, a field programmable gate array (FPGA) chip, and the like. In a case where a plurality of received signals are output from the acoustic wave receiving unit 17, it may be desirable that the data acquisition unit 19 is capable of simultaneously processing the plurality of signals. This results in a reduction in time used to acquire object information. In the present specification, regarding the “received signal”, the analog signal output from the acoustic wave receiving unit 17 and the digital signal obtained by performing the analog-to-digital conversion on the analog signal both fall in the scope of the “received signal”.

### (Computer 20)

The computer 20 is typically a workstation, a large-scale parallel cluster, or the like, and executes all processes on the received signals by preprogrammed software. Note that the computer 20 may execute part or all of processes by hardware instead of software on the workstation or the like. In the present embodiment, the processes may be executed individually by other apparatuses instead of being all executed by the computer 20.

The computer 20 includes a processing unit 20a capable of performing a particular process on the electric signal output from the acoustic wave receiving unit 17. The processing unit 20a as a control unit is capable of controlling operations of the respective elements of the photoacoustic apparatus via a bus 30 as illustrated in FIG. 2.

The processing unit 20a includes typically elements such as a CPU, a GPU, an analog-to-digital converter and/or the like and/or a circuit such as a FPGA and/or an application specific integrated circuit (ASIC). The processing unit 20a may be formed using one element or circuit or may be formed using a plurality of elements or circuits. Each process may be performed by any element or circuit in the processing unit 20a.

The computer 20 further includes a storage unit 20b including a storage medium which may be typically a ROM, a RAM, a hard disk, or the like. The storage unit 20b may include only one storage medium or may include a plurality of storage media.

It may be desirable to configure the computer 20 so as to be capable of performing a plurality of processes in parallel by pipelining or the like. This results in a reduction in time used to acquire object information.

Programs executed by the computer 20 in terms of the signal processing and/or the controlling of the operation of the photoacoustic apparatus may be stored in the storage unit 20b. Note that when the programs are stored in the storage unit 20b, a non-temporary storage medium is used.

Depending on the situation, the data acquisition unit 19 and the computer 20 may be integrated together. In this case, image data of an object may be generated by performing a process using hardware instead of software such as that performed by a workstation. Note that the data acquisition unit 19 and the computer 20 may be generically referred to as a processing unit in the present specification.

### (Display Apparatus 21)

The display apparatus 21 is an apparatus configured to display the image data of the object information output from the computer 20 such that the object information is displayed in the form of an image or numerical information. The display apparatus 21 may be typically a liquid crystal display or the like. Note that the display apparatus 21 may be provided separately from the photoacoustic apparatus according to the present embodiment.

### <Method of Operating Photoacoustic Apparatus>

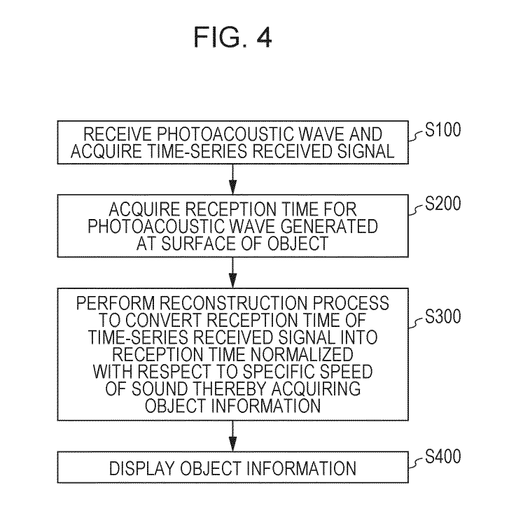

An operation process of the photoacoustic apparatus illustrated in FIG. 1 according to the present embodiment is described below referring also to FIG. 4. Note that processing numbers described below correspond to processing numbers illustrated in FIG. 4.

### (S100: Receiving Photoacoustic Wave and Acquiring Time-Series Received Signal)

First, the light source 11 generates light 12 and the object 15 is illuminated with the light 12 via the optical system 13. The light 12 is absorbed by the light absorbent 14 located inside the object 15. The absorption of the light 12 causes the light absorbent 14 to expand instantaneously. As a result, the photoacoustic waves 16a and 16b are generated.

The acoustic wave receiving unit 17 receives the photoacoustic waves 16a and 16b and converts them into time-series received signals. The data acquisition unit 19 performs the amplification and the analog-to-digital conversion on the received signals output from the acoustic wave receiving unit 17 and stores the resultant time-series received signals in the form of digital signals in the storage unit 20b. In the present embodiment, in synchronization with the timing of generating the light 12 by the light source 11, the data acquisition unit 19 starts the above-described process on the received signal output from the acoustic wave receiving unit 17.

### (S200: Acquiring Reception Time of Photoacoustic Wave Generated on Surface of Object)

Based on the time-series received signals acquired in S100, the processing unit 20a calculates the time tₐ at which the photoacoustic wave 16a generated on the surface 22 of the object 15 is received by the acoustic wave receiving unit 17. In the present embodiment, the light 12 emitted from the optical system 13 hits the surface 22 of the object 15 from the side of the acoustic wave receiving unit 17. Therefore, the photoacoustic wave 16a generated on the surface 22 of the object 15 is received first of all photoacoustic waves generated by the object 15. That is, of time-series received signals illustrated in FIG. 3A, the signal A is a received signal corresponding to the photoacoustic wave generated on the surface 22 of the object 15.

For example, in a case where the signal A includes noise, it is possible to distinguish the received signal from the noise by using the property that the received signal has a shape close to the shape of impulse response of the acoustic wave receiving unit 17. That is, the processing unit 20a performs pattern matching on the time-series received signals with respect to the impulse response of the acoustic wave receiving unit 17 and extracts, as the received signal, a signal that matches the pattern of the impulse response. Furthermore, the processing unit 20a detects a signal received first from received signals extracted via the pattern matching, and employs the detected signal as the received signal of the photoacoustic wave generated on the surface 22 of the object 15.

The surface 22 of the object 15 has a high light illumination intensity and is close in distance to the acoustic wave receiving unit 17, and thus the sound pressure received by the acoustic wave receiving unit 17 is generally greater than the sound pressure for optoacoustic signals generated by other light absorbents 14 in the object 15. By using the above feature, the processing unit 20a determines that a greatest signal in the time-series received signals is a received signal corresponding to the photoacoustic wave generated on the surface 22 of the object 15.

As described above, the photoacoustic wave generated on the surface 22 of the object 15 has a feature different from that of a photoacoustic wave generated by another light absorbent 14. Therefore, based on the time-series received signals, the processing unit 20a is capable of acquiring the reception time of the photoacoustic wave generated on the surface 22 of the object 15 by using any extraction method based on the feature of the photoacoustic wave generated on the surface 22 of the object 15. This method allows it to acquire the reception time of the photoacoustic wave generated on the surface of the object without increasing the complexity of the apparatus.

Note that any other methods may also be employed as long as the methods are capable of acquiring the reception time of the photoacoustic wave generated on the surface 22 of the object 15. For example, the reception time of the photoacoustic wave generated on the surface 22 of the object 15 may be estimated from received signals of reflected waves of ultrasonic waves transmitted from an ultrasonic wave transmission unit. Alternatively, an apparatus configured to acquire coordinates on the surface of the object 15 may be used to determine the distance between the acoustic wave receiving unit 17 and the surface 22 of the object 15, and the reception time of the photoacoustic wave generated on the surface 22 of the object 15 may be estimated based on the distance.

(S300: Acquiring Subject Information by Performing Reconstruction Process to Convert Reception Time of Time-Series Received Signal to Reception Time Normalized with Respect to Particular Speed of Sound)

The processing unit 20a acquires the object information by performing the reconstruction process on the time-series received signals stored in S200 in the storage unit 20b using the speed of sound inside the object 15 as the specific speed of sound. In this reconstruction process, the processing unit 20a converts the reception time such that the reception time t1 of the time-series received signal acquired in S200 by the signal processing method described above is converted to the reception time t2 normalized with respect to the speed of sound inside the object 15. This makes it possible to obtain subject information while suppressing the influence of the difference between the speed of sound in the object 15 and the speed of sound in the acoustic matching material 18.

As for an algorithm of reconstructing values of particular smallest constituent units from a plurality of time-series received signals, for example, a back projection method may be used in a time domain or a Fourier domain using a specific speed of sound commonly employed in tomography techniques. In a case where it is allowed to spend much time to perform the reconstruction process, the reconstruction may be performed using an inverse problem solving algorithm using an iteration process. In photoacoustic tomography, which is one of photoacoustic imaging techniques, the reconstruction may be performed using various techniques. Typical examples of techniques are a Fourier transform method, a universal back projection method, and a filtered back projection method (“Photoacoustic imaging in biomedicine”, M. Xu, L. V. Wang, REVIEW OF SCIENTIFIC INSTRUMENT, 77, 041101, 2006).

The processing unit 20a may acquire a light fluence distribution of the light 12 in the object 15. The processing unit 20a may acquire, as object information, an absorption coefficient distribution in the object 15 by correcting the initial sound pressure distribution with respect to the light fluence distribution. The absorption coefficient distribution may be acquired for a plurality of different wavelengths by performing the process S100 to S400 for light with the respective different wavelengths. A concentration distribution of a substance may be acquired as object information by using the absorption coefficient distribution for a plurality of wavelengths.

### (S400: Displaying Object Information)

The processing unit 20a outputs the object information obtained in S300 to the display apparatus 21 to display the object information in the form of an image or numerical information on the display apparatus 21.

By performing the process described above, it is possible to obtain object information while suppressing the influence of the difference between the speed of sound in the object and the speed of sound in the acoustic matching material in the case where the acoustic matching material is disposed between the object and the acoustic wave receiving unit.

### First Example

An example of a photoacoustic apparatus according to an embodiment is described below with reference to FIG. 1.

In the present example, Ti:Sa laser system pumped by a second-order-harmonic YAG laser is used as the light source 11. Using the Ti:Sa laser, it is possible to illuminate an object with light with a wavelength in a range from 700 to 900 nm. The laser light is passed through the optical system 13 including a mirror, a beam expander, and the like to obtain a radius expanded to about 1 cm, and the surface 22 of the object 15 is illuminated with the laser light with the expanded beam diameter from the side of the acoustic wave receiving unit 17.

A piezoelectric probe including a two-dimensional array of 15×23 elements is used as the acoustic wave receiving unit 17.

The data acquisition unit 19 has a function of simultaneously receiving all data of 345 channels from the acoustic wave receiving unit and transferring the received data, after amplifying and converting analog data to digital data, to the computer 20. The sampling frequency of the data acquisition unit 19 is set to 20 MHz, and the data acquisition unit 19 starts receiving the data in synchronization of the start of illuminating the object with light.

A hemispherical phantom mimicking a living body is used as the object 15. The phantom is made of urethane rubber including a mixture of titanium oxide functioning as a scattering material and ink functioning as an absorber material. A spherical black rubber with a diameter of 0.5 mm is embedded as the light absorbent 14 in the center of the hemispherical urethane phantom. The phantom has a diameter of 40 mm. The urethane phantom is in contact with the acoustic wave receiving unit 17 via a transparent gel pad functioning as the acoustic matching material 18. The shape of the gel pad is capable of being flexibly changed according to the shape of the phantom. The distance between the surface of the phantom and the acoustic wave receiving unit 17 is set to about 30 mm. The speed of sound c_{b} in the urethane phantom is 1409 m/s and the speed of sound cₐ in the gel pad used as the acoustic matching material 18 is 1490 m/s, and thus there is a difference in speed of sound.

First, the phantom is illuminated with light with a wavelength of 756 nm emitted from the Ti:Sa laser. Time-series received signals obtained as a result are stored in the storage unit 20b (S100). The obtained received signals are schematically illustrated in FIG. 3A.

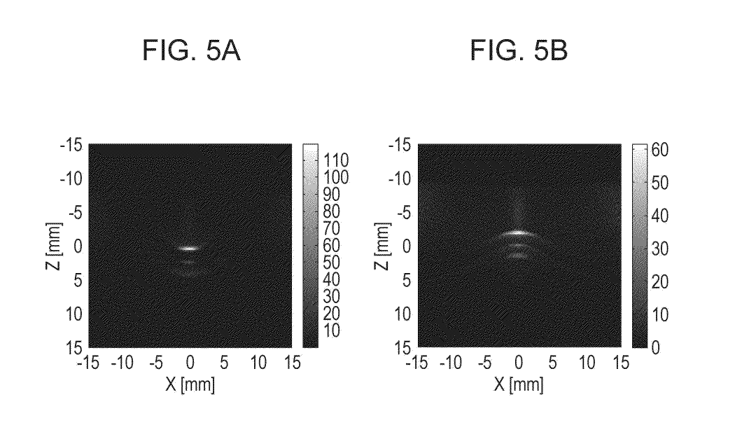

For comparison, the processing unit 20a performs reconstruction process on the time-series received signals using the speed of sound c_{b} in the phantom. More specifically, the processing unit 20a performs the reconstruction process according to equation (7) which is an example of a time-domain reconstruction process thereby obtaining a reconstructed image representing an initial sound pressure distribution p₀(r) as illustrated in FIG. 5B.

$${p}_{0} ( r ) \approx - \frac{1}{2 π} {\int }_{{S}_{0}} \int {\frac{d {S}_{0}}{{r}_{0}^{2}} [ t 1 \frac{\partial {p}_{d} ( {r}_{0} , t 1 )}{\partial t 1} + 2 {p}_{d} ( {r}_{0} , t 1 ) ]}_{t 1 = \vert r - {r}_{0} \vert / {c}_{b}} \qquad ( 7 )$$

In equation (7), r denotes a position vector of a reconstructed voxel, S₀ denotes a receiving area size of each element in the acoustic wave receiving unit 17, r₀ denotes a position vector of each element in the acoustic wave receiving unit 17, and p_{d} denotes a time-series received signal.

Next, the processing unit 20a calculates the reception time tₐ of the photoacoustic wave generated on the surface of the urethane phantom from the time-series received signals (S200). In the present example, a correlation value between each time-series received signal and the impulse response of the acoustic wave receiving unit 17 is calculated, and a signal received first of signals having high correlation coefficients is determined as the received signal of the photoacoustic wave generated on the surface of the urethane phantom. More specifically, 20.2 microseconds is obtained as the reception time for the signal determined as the received signal of the photoacoustic wave generated on the surface of the urethane phantom, and this reception time is denoted by tₐ.

Next, the processing unit 20a performs the reconstruction process on the time-series received signals stored in the storage unit 20b by using the speed of sound c_{b} in the phantom (S300). More specifically, the processing unit 20a performs the reconstruction process by treating the time-series received signals such that the reception time t1 of each piece of time-sampled data in the period until the time tₐ is multiplied by cₐ/c_{b}. Furthermore, in the reconstruction process, the processing unit 20a treats the time-series received signals such that the reception time t1 of each piece of time-sampled data in the period after the time tₐ is given by tₐ×cₐ/c_{b}+(t1−tₐ).

In the present example, cₐ/c_{b}=1490/1409=1.057. That is, the processing unit 20a converts the reception time t1 of each time-sampled data in the period until the time tₐ to a new reception time t2 according to an equation t2=t1×1.057. On the other hand, for each time-sampled data in the period after the time tₐ, the reception time t1 is converted to a new reception time t2 according to an equation t2=20.2 seconds×1.057+(t1−20.2 seconds). Furthermore, the processing unit 20a performs the reconstruction process using data of the converted reception time t2, the time-series received signals stored in the storage unit 20b, and the speed of sound c_{b} in the phantom. In the present example, more specifically, the processing unit 20a performs the reconstruction process according to equation (8) expressed by the reception time t2 obtained by converting the reception time t1 in equation (7). As a result, a reconstructed image representing an initial sound pressure distribution p₀(r) is obtained as illustrated in FIG. 5A.

$${p}_{0} ( r ) \approx - \frac{1}{2 π} {\int }_{{S}_{0}} \int {\frac{d {S}_{0}}{{r}_{0}^{2}} [ t 2 \frac{\partial {p}_{d} ( {r}_{0} , t 2 )}{\partial t 2} + 2 {p}_{d} ( {r}_{0} , t 2 ) ]}_{t 2 = \vert r - {r}_{0} \vert / {c}_{b}} \qquad ( 8 )$$

FIG. 5A is compared below with FIG. 5B. FIG. 5A and FIG. 5B both represent a two-dimensional cross section view taken near the center of the phantom. In FIG. 5B, an image of the light absorbent 14 in the urethane phantom is located at a position different from the true position (the center of the phantom), and the image in FIG. 5B is worse in terms of resolution and contrast than in FIG. 5A. On the other hand, in FIG. 5A, the image of the light absorbent 14 is located at the true position of the light absorbent 14, and the image is sharper than in FIG. 5B.

In the present example, as described above, it is possible to obtain object information while suppressing the influence of the difference between the speed of sound in the object and the speed of sound in the acoustic matching material without increasing the complexity of the apparatus even in a case where the shape of the surface of the object is not known.

### Second Example

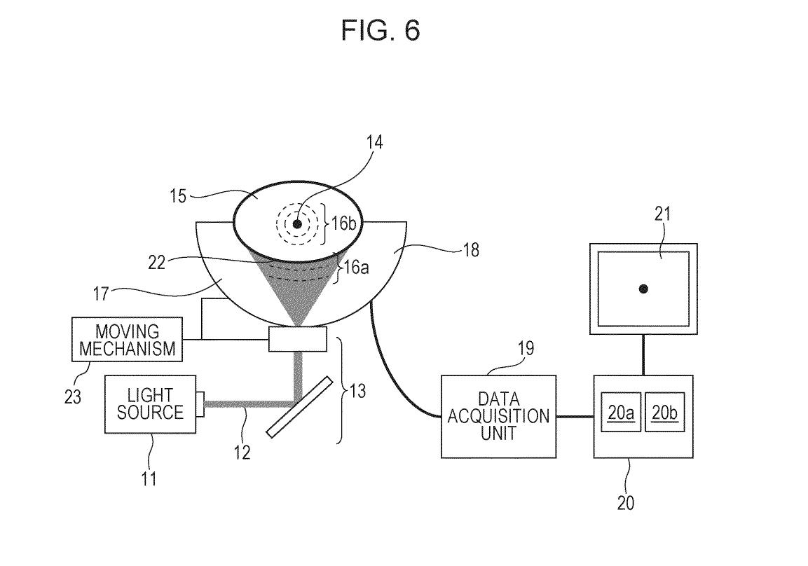

An example of a photoacoustic apparatus according to an embodiment is described below with reference to FIG. 6. Elements similar to those in FIG. 1 are basically denoted by similar reference numerals, and a further description thereof is omitted.

The second example is different from the first example in that the conversion of the reception time is performed only for time-sampled data in the period after the reception time tₐ of the photoacoustic wave generated on the surface of the phantom.

The second example is also different from the first example in that a moving mechanism 23 is provided to move the acoustic wave receiving unit 17 relative to the object 15. The provision of the moving mechanism 23 makes it possible to change the reception position of a photoacoustic wave such that the photoacoustic wave is received at a plurality of positions. Note that the moving mechanism 23 also moves the optical system 13 in synchronization of the movement of the acoustic wave receiving unit 17. Note that the moving mechanism 23 is driven under the control of the processing unit 20a.

As described above, when the distance between the surface 22 of the object 15 and the light absorbent in the object is denoted by d_{b1} and the distance between the surface 22 of the object 15 and the acoustic wave receiving unit 17 is denoted by d_{b2}, it may be desirable to set the positional relationship between the object 15 and the acoustic wave receiving unit 17 such that d_{b1}<d_{b2}. That is, it may be desirable to set the positional relationship between the object 15 and the acoustic wave receiving unit 17 so as to obtain high accuracy of approximate expression (3). To achieve the above situation, in the present example, the moving mechanism 23 moves the acoustic wave receiving unit 17 such that the acoustic wave receiving unit 17 is capable of receiving photoacoustic waves at a position that satisfies d_{b1}<d_{b2}. Furthermore, the moving mechanism 23 moves the acoustic wave receiving unit 17 such that the acoustic wave receiving unit 17 satisfies d_{b1}<d_{b2} at any reception position. More specifically, in the present example, the condition d_{b1}<d_{b2} is achieved by controlling the position of the acoustic wave receiving unit 17 such that each receiving element of the acoustic wave receiving unit 17 is apart from the surface of the phantom by a distance equal to or greater than 50 mm.

In the present example, an alexandrite laser which is a solid-state laser capable of emitting light with a wavelength of 755 nm is employed as the light source 11. A phantom is a hemisphere-shaped urethane phantom same as that used in the first example. The acoustic wave receiving unit 17 is configured such that 512 receiving elements are disposed in a spiral manner on the surface of a hemisphere. Water is disposed as the acoustic matching material 18 in the hemisphere-shaped acoustic wave receiving unit 17 such that the phantom is in contact with the acoustic wave receiving unit 17 via the water. Note that the water used as the acoustic matching material 18 is liquid and thus the shape of the acoustic matching material 18 is allowed to freely change according to the shape of the phantom.

First, light with a wavelength of 755 nm is emitted from the alexandrite laser. Time-series received signals obtained as a result are stored in the storage unit 20b (S100).

Next, the reception time tₐ of the photoacoustic wave generated on the surface of the urethane phantom is calculated from the time-series received signals (S200). In the present example, a correlation value between each time-series received signal and the impulse response of the acoustic wave receiving unit 17 is calculated, and a signal received first of signals having high correlation coefficients is determined as the received signal of the photoacoustic wave generated on the surface of the urethane phantom. More specifically, 42.3 microseconds is obtained as the reception time for the signal determined as the received signal of the photoacoustic wave generated on the surface of the urethane phantom, and this reception time is denoted by tₐ.

Next, the processing unit 20a preforms the reconstruction process on the time-series received signals stored in the storage unit 20b by using the speed of sound cₐ in the acoustic matching material 18 (S300). In this reconstruction process, the processing unit 20a treats the time-series received signals such that the reception time t1 of each piece of time-sampled data in the period after the time tₐ is given by an equation t2=tₐ+(t1−tₐ)×c_{b}/cₐ.

In the present example, data is sampled at a sampling frequency of 20 MHz, and the total number of sampling points is 3048. When the received signal is sampled at a sampling frequency of 20 MHz, 846 sampling points are obtained in a period until a reception time of 42.3 microseconds at which the received signal of the photoacoustic wave generated at the surface of the urethane phantom is received, and as many sampling points as 3048−846=2202 are obtained for the following period. Note that c_{b}/ca=1409/1490=0.946. Thus, the processing unit 20a generates reception time t2 for each of 847th to 3048th sampling points by converting the reception time t1 to t2 according to an equation t2=42.3 microseconds+(t1−42.3 microseconds)×0.946. Furthermore, in the reconstruction process, the processing unit 20a performs the reconstruction process using data of the converted reception time t2, the time-series received signals stored in the storage unit 20b, and the speed of sound cₐ in the acoustic matching material 18. More specifically, in the present example, the processing unit 20a performs the reconstruction process according to equation (9). As a result, a reconstructed image illustrated in FIG. 5A is obtained.

$${p}_{0} ( r ) \approx - \frac{1}{2 π} {\int }_{{S}_{0}} \int {\frac{d {S}_{0}}{{r}_{0}^{2}} [ t 2 \frac{\partial {p}_{d} ( {r}_{0} , t 2 )}{\partial t 2} + 2 {p}_{d} ( {r}_{0} , t 2 ) ]}_{t 2 = \vert r - {r}_{0} \vert / {c}_{a}} \qquad ( 9 )$$

In the present example, the photoacoustic apparatus may include a notification unit configured to visually or aurally notify a user whether the condition d_{b1}<d_{b2} is satisfied or not for a determined moving range of the acoustic wave receiving unit 17. For example, the display apparatus 21 may be used as the notification unit, and information indicating whether the condition d_{b1}<d_{b2} is satisfied or not may be displayed on the display apparatus 21 thereby providing the notification to a user. Alternatively, for example, a lamp may be used as the notification unit and information indicating whether the condition d_{b1}<d_{b2} is satisfied or not may be indicated by a color of the lamp thereby providing the notification to a user. Alternatively, for example, a speaker may be used as the notification unit and information indicating whether the condition d_{b1}<d_{b2} is satisfied or not may be indicated by a sound generated by the speaker thereby providing the notification to a user.

As described above, the notification unit allows a user to know whether the condition d_{b1}<d_{b2} is satisfied or not. Thus, for example, when the user recognized that the condition d_{b1}<d_{b2} is not satisfied, the user may reset the moving range of the acoustic wave receiving unit 17 such that the condition d_{b1}<d_{b2} is satisfied.

In the present example, as described above, the reception time tₐ of the photoacoustic wave generated on the surface of the object is detected from the time-series received signal, and the reception time of each time-sampled data in the period after tₐ is corrected. In the present example, it is possible to obtain object information while suppressing the influence of the difference between the speed of sound in the object and the speed of sound in the acoustic matching material without increasing the complexity of the apparatus even in a case where the shape of the surface of the object is not known. Furthermore, in the present example, the position of the acoustic wave receiving unit is controlled by the moving mechanism so as to obtain high accuracy of approximate expression (3), and thus it is possible to obtain object information with high accuracy using t_{b} represented by equation (3).

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

Specific embodiments have been described above. Note that the present invention is not limited to the embodiments described above, but various modifications and applications are possible without departing from the scope of the invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-269691 filed Dec. 26, 2013, which is hereby incorporated by reference herein in its entirety.

What is claimed is:

1. An apparatus comprising:
a receiving unit configured to receive a photoacoustic wave that is generated when an object is illuminated with light and to output a time-series received signal; and
a processing unit configured to acquire object information by performing a reconstruction process on the time-series received signal using a specific speed of sound,
wherein in the reconstruction process, the processing unit converts a reception time of the time-series received signal to a reception time normalized with respect to the specific speed of sound.

2. The apparatus according to claim 1, wherein the processing unit converts the reception time of the time-series received signal before or after a reception time of a photoacoustic wave generated on a surface of the object such that the reception time of the time-series received signal is normalized with respect to the specific speed of sound.

3. The apparatus according to claim 2, wherein the processing unit acquires, from the time-series received signal, the reception time of the photoacoustic wave generated on the surface of the object.

4. The apparatus according to claim 1, wherein the processing unit converts the reception time of the time-series received signal to the reception time normalized with respect to the specific speed of sound by using a speed of sound in the object and a speed of sound in a material disposed between the object and the receiving unit.

5. The apparatus according to claim 4, the processing unit converts the reception time of the time-series received signal to the reception time normalized with respect to the specific speed of sound by using a ratio of the speed of sound in the object to the speed of sound in the material and the reception time of the photoacoustic wave generated on the surface of the object.

6. The apparatus according to claim 1, wherein the specific speed of sound is the speed of sound in the object.

7. The apparatus according to claim 1, wherein the specific speed of sound is the speed of sound in the material disposed between the object and the receiving unit.

8. The apparatus according to claim 7, wherein the material disposed between the object and the receiving unit is a material having an acoustic impedance between an acoustic impedance of the object and an acoustic impedance of the receiving unit.

9. A method comprising:
acquiring object information by performing a reconstruction process, using a specific speed of sound, on a time-series received signal obtained by receiving a photoacoustic wave, from a receiving unit, generated when an object is illuminated with light,
wherein the acquiring the object information includes converting a reception time of the time-series received signal to a reception time normalized with respect to a specific speed of sound.

10. The method according to claim 9, wherein the converting includes the reception time of the time-series received signal before or after a reception time of a photoacoustic wave generated on a surface of the object such that the reception time of the time-series received signal is normalized with respect to the specific speed of sound.

11. The method according to claim 10, further comprising acquiring, from the time-series received signal, the reception time of the photoacoustic wave generated on the surface of the object.

12. The method according to claim 9, wherein the converting includes converting the reception time of the time-series received signal to the reception time normalized with respect to the specific speed of sound by using a speed of sound in the object and a speed of sound in a material disposed between the object and the receiving unit.

13. The method according to claim 9, wherein the specific speed of sound is the speed of sound in the object.

14. The method according to claim 9, wherein the specific speed of sound is the speed of sound in the material disposed between the object and the receiving unit.

15. A program that causes a computer to execute method comprising:
acquiring object information by performing a reconstruction process, using a specific speed of sound, on a time-series received signal obtained by receiving a photoacoustic wave, from a receiving unit, generated when an object is illuminated with light,
wherein the acquiring the object information includes converting a reception time of the time-series received signal to a reception time normalized with respect to a specific speed of sound.

16. The program according to claim 15, wherein the converting includes the reception time of the time-series received signal before or after a reception time of a photoacoustic wave generated on a surface of the object such that the reception time of the time-series received signal is normalized with respect to the specific speed of sound.

17. The program according to claim 16, further comprising acquiring, from the time-series received signal, the reception time of the photoacoustic wave generated on the surface of the object.

18. The program according to claim 15, wherein the converting includes converting the reception time of the time-series received signal to the reception time normalized with respect to the specific speed of sound by using a speed of sound in the object and a speed of sound in a material disposed between the object and the receiving unit.

19. The program according to claim 15, wherein the specific speed of sound is the speed of sound in the object.

20. The program according to claim 15, wherein the specific speed of sound is the speed of sound in the material disposed between the object and the receiving unit.
